(19) United States
(12) Patent Application Publication
(10) Pub. No.: US 2008/0097151 A1
(43) Pub. Date: Apr. 24, 2008

(54) ENDOSCOPE PROCESSOR AND ENDOSCOPE SYSTEM

(75) Inventors: Takuya INOUE, Saitama (JP); Nobuo ARIKAWA, Tokyo (JP); Nobuhito NAKAYAMA, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/874,424

(22) Filed: Oct. 18, 2007

(30) Foreign Application Priority Data
Oct. 19, 2006 (JP) 2006-284970

(51) Int. Cl. A61B 1/005

(52) U.S. Cl. 600/109; 348/65

(57) ABSTRACT

An endoscope processor comprising a touch-panel monitor, a location detector, a touch-panel image generator, and a location-changer, is provided. The endoscope processor displays an image of a target area with enlargement on a monitor. The target area is a part of a captured entire image. The location detector detects an input location. The input location is a location where the user's input operation is done on the touch-panel monitor. The touch-panel image generator orders a target-area location window to be displayed on the touch-panel monitor. The target-area location window indicates the location of the target area in the entire image. The location-changer changes the location of the target area based on the input location detected by the location detector when the target-area location window is displayed.

### BACKGROUND OF THE INVENTION

1. Field of the Invention

The present invention relates to an endoscope processor that displays an image captured by an electronic endoscope and enlarged a part of the image.

2. Description of the Related Art

By carrying out specified signal processing on the image signal generated by an electronic endoscope, various images can be displayed on a monitor. Japanese Patent Publication No. 2001-137183 discloses that a part of an image captured by an electronic endoscope is enlarged and displayed, the location of the image to be enlarged is changed according to the user's command input, and the location of the enlarged area relative to the whole image is displayed with a part of the enlarged image.

The user can change the location to be enlarged in an image by keyboard input. Therefore, the user must input with the keyboard while watching a part of the enlarged image displayed on the monitor. It is inconvenient to input with a keyboard while watching a monitor.

### SUMMARY OF THE INVENTION

Therefore, an object of the present invention is to provide an endoscope processor that carries out signal processing on the image signal so that a part of the entire image captured by an imaging device may be enlarged and displayed, and a user may easily and comfortably change the location to be enlarged in.

According to the present invention, an endoscope processor comprising a touch-panel monitor, a location detector, a touch-panel image generator, and a location-changer, is provided. The endoscope processor displays an image of a target area with enlargement on a monitor. The target area is a part of an entire image captured by an electronic endoscope. The location detector detects an input location. The input location is a location where the user's input operation is done on the touch-panel monitor. The touch-panel image generator orders a target-area location window to be displayed on the touch-panel monitor. The target-area location window indicates the location of the target area in the entire image. The location-changer changes the location of the target area based on the input location. The input location is detected by the location detector when the target-area location window is displayed on the touch-panel monitor.

Further, when the location-changer changes the location of the target area, the touch-panel image generator renews the target-area location window based on the changed location of the target area.

Further, the entire image is displayed on the touch-panel monitor. The target-area location window is the displayed entire image where the location of the target area is indicated.

Further, the location change clock changes the location of the target area so that the center of the target area agrees with the input location.

Further, it is permitted to change the location of the target area within a permission area. The extent of the permission area is decided according to the magnification factor used to enlarge an image of the target area.

### BRIEF DESCRIPTION OF THE DRAWINGS

The objects and advantages of the present invention will be better understood from the following description, with reference to the accompanying drawings in which:

FIG. 1 is a perspective view of an endoscope system having an endoscope processor which is an embodiment of the present invention;

FIG. 2 is a block diagram showing the internal structure of the electronic endoscope and the endoscope processor;

FIG. 3 illustrates a zooming-adjustment command-input picture;

FIG. 4 shows the locations of a first, second, and third areas, and a permission area in the zooming-adjustment command-input picture;

FIG. 5 shows unit areas in the first area; and

FIG. 6 is a flowchart describing the image displaying process as carried out by the endoscope processor.

### DESCRIPTION OF THE PREFERRED EMBODIMENTS

The present invention is described below with reference to the embodiments shown in the drawings.

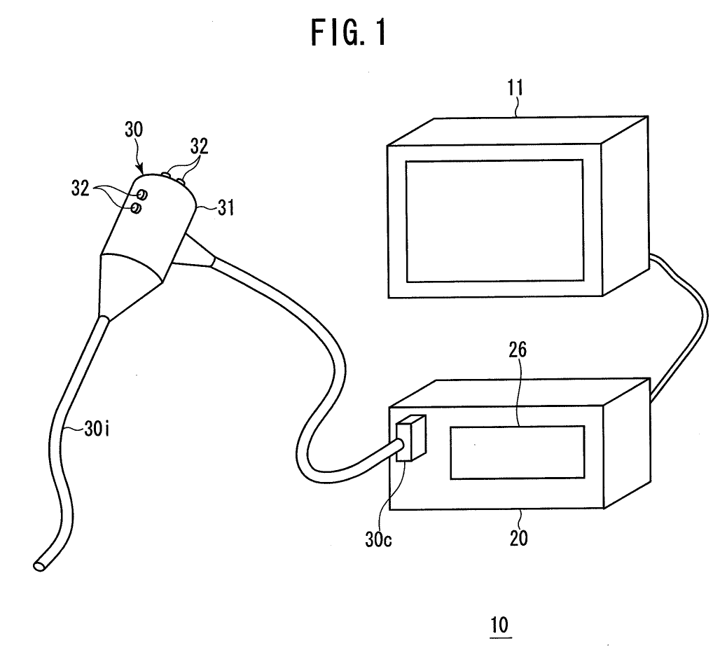

In FIG. 1, an endoscope system 10 comprises an endoscope processor 20, an electronic endoscope 30, and a monitor 11. The electronic endoscope 30 is connected to the endoscope processor 20 via the connector 30c. The monitor 11 is connected to the endoscope processor 20 via a connector (not depicted).

The whole structure of the endoscope system 10 is briefly explained. A light-source unit (not depicted in FIG. 1) which is housed in the endoscope processor 20 supplies light to the electronic endoscope 30. The supplied light is transmitted to the head end of an insertion tube 30i and illuminates a peripheral area around the head end of the insertion tube 30i of the electronic endoscope 30. An optical image of the illuminated subject is captured by an imaging device (not depicted in FIG. 1), such as a CCD image sensor, mounted at the head end of the insertion tube 30i.

Subsequently, an image signal corresponding to the image of the captured subject is generated by the imaging device. The image signal is sent to the endoscope processor 20, where predetermined signal processing is carried out on the image signal. The image signal, having undergone the predetermined signal processing, is sent to the monitor where the resulting image is displayed.

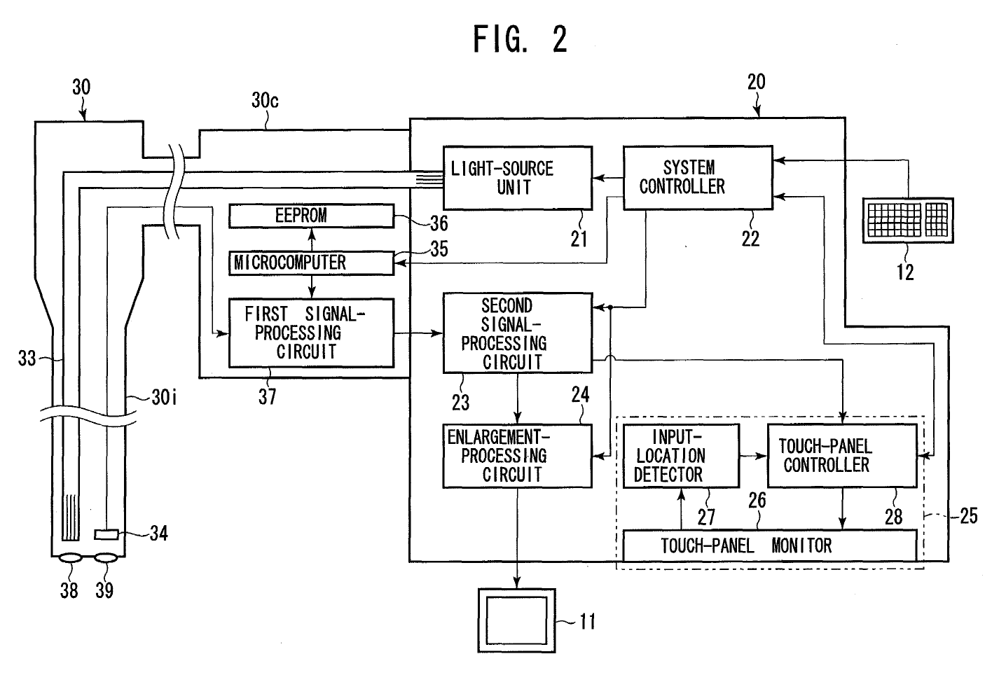

Next, an internal mechanism of the electronic endoscope 30 and the endoscope processor 20 is explained in detail with reference to FIG. 2.

The electronic endoscope 30 comprises a light guide 33, an imaging device 34, a microcomputer 35, an electronically erasable programmable ROM (EEPROM) 36, a first signal-processing circuit 37, and other components.

The light guide 35 is a bundle of optical fibers, of which one end, hereinafter referred to as the incident end, is mounted in the connector 30c and the other end, hereinafter referred to as the exit end, is mounted in the head end of the insertion tube 30i. Illumination light incident on the incident end is transmitted to the exit end. The illumination light transmitted to the exit end illuminates a peripheral area around the head end of the insertion tube 30i after passing through a diffuser lens 38.

An optical image of the illuminated subject is focused onto the light-receiving surface of the imaging device 34 by an object lens 39. The imaging device 34 generates an image signal corresponding to the optical image focused onto the light-receiving surface. The image signal is sent to the first signal-processing circuit 37 housed in the connector 30c.

The first signal-processing circuit 37 carries out predetermined signal processing on the received image signal. The image signal, having undergone predetermined signal processing, is sent to the endoscope processor 20. Incidentally, the microcomputer 35 controls some components of the entire electronic endoscope 30 in order to carry out some operations, such as predetermined signal processing carried out by the first signal-processing circuit 37, and the release operation by the imaging device 34. Necessary data for the control of the microcomputer 35 is stored in the EEPROM 36.

The endoscope processor 20 comprises the light-source unit 21, a system controller 22, a second signal-processing circuit 23, an enlargement-processing circuit 24 (location-changer), a touch-panel input unit 25, and other components.

When the electronic endoscope 30 is connected to the endoscope processor 20, the light guide 33 is optically connected to the light-source unit 21. When the user observes an subject with the electronic endoscope 30, light emitted by the light-source unit 21 is supplied to the incident end of the light guide 33 as illumination light.

In addition, when the electronic endoscope 30 is connected to the endoscope processor 20, the first and second signal-processing circuits 37 and 23 are electrically connected to each other. The image signal output from the first signal-processing circuit 37 is input to the second signal-processing circuit 23. The second signal-processing circuit 23 carries out predetermined signal processing on the received image signal.

The image signal, on which the second signal-processing circuit 23 has carried out predetermined signal processing, is sent to the enlargement-processing circuit 24. As described later, a part of an entire captured image can be displayed while being enlarged by the endoscope system 10. For displaying with enlargement, the enlargement-processing circuit 24 carries out enlargement processing on a partial signal component of the image signal. Either the image signal or the partial signal component having undergone enlargement processing is sent to the monitor 11, where it is displayed.

The system controller 22 controls the light-source unit 21, the second signal-processing circuit 23, and the enlargement-processing circuit 24, ordering them on or off; the latter two in order to carry out predetermined signal processing and to carry out enlargement processing, respectively. The system controller 22 controls those components according to the user's command input to keyboard 12 or the touch-panel input unit 25.

The touch-panel input unit 25 comprises a touch-panel monitor 26, an input-location detector 27, and a touch-panel controller 28 (touch-panel image generator).

The touch-panel monitor 26 is mounted on a front face of the endoscope processor 20 (see FIG. 1). On the touch-panel monitor 26, a command-input picture is displayed. When a user touches the picture of a virtual button in the command-input picture, the endoscope system 10 commences to carry out a function according to the touched button.

The touch-panel controller 28 generates data of the command-input picture. The data of the command-input picture is generated under the control of the system controller 22 based on data sent from the second signal-processing circuit 23.

The input-location detector 27 detects the location where the user touched the touch-panel monitor 26, hereinafter referred to as touched location (input location). The input-location detector 27 generates a location signal corresponding to the touched location. The location signal is sent to the touch-panel controller 28. The touch-panel controller 28 generates an input-command signal based on the currently-displayed command-input picture and the location signal. The input-command signal is sent to the system controller 22.

Next, displaying an enlarged image is explained. When observing an entire image, the entire optical image captured by the effective pixel area of the imaging device 34 is displayed on the monitor 11. On the other hand, when observing an enlarged image, a part of the image captured by a partial area of the effective pixel area displayed on the monitor 11 is enlarged.

Display of an entire image and an enlarged image alternate according to a command input to a zooming-adjustment lever (not depicted) mounted on the electronic endoscope 30 or the touch-panel input unit 25.

When displaying an entire image, a general command-input picture, including a light button for switching on and off the light-source unit 21, a brightness-adjustment button, a color-balance adjustment button, and a menu-change button, is displayed on the touch-panel monitor 26.

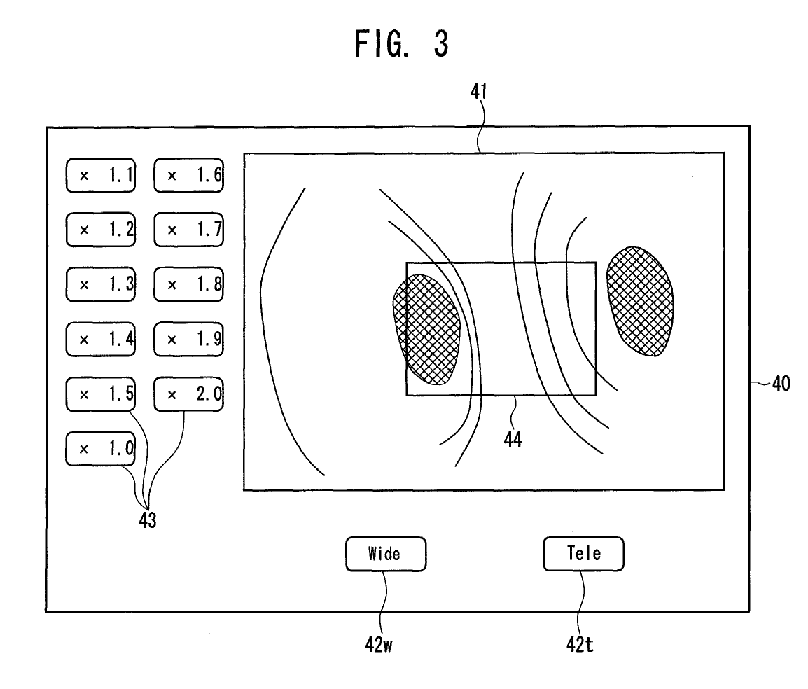

When the menu-change button is touched, a menu-command input picture, including various kinds of virtual buttons to carry out corresponding functions, and an enlarged-image display button, is displayed on the touch-panel monitor 26. When the enlarged-image display button is touched, a zooming-adjustment command-input picture 40, as shown in FIG. 3, is displayed on the touch-panel monitor 26.

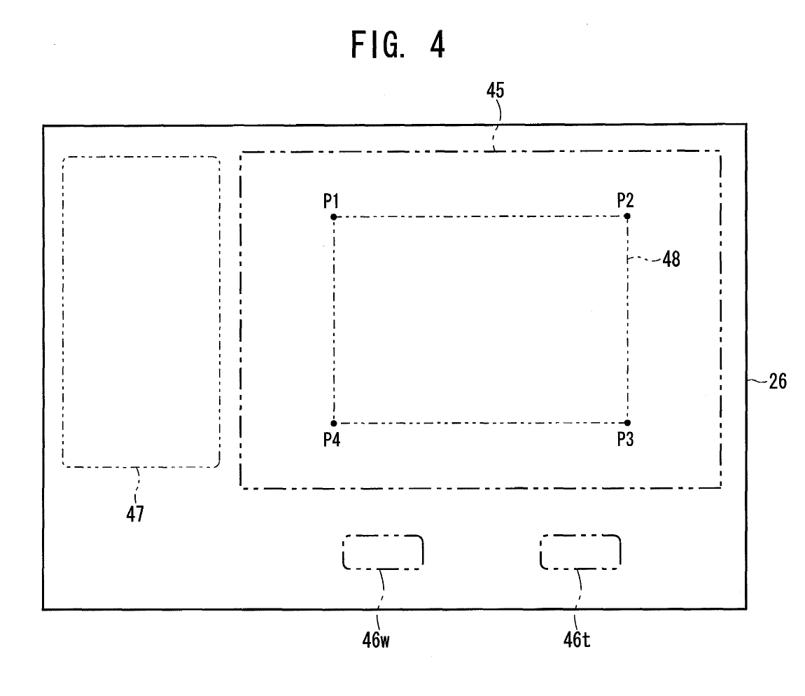

The zooming-adjustment command-input picture 40 includes an enlarged-area location map 41 (target-area location window), zooming-adjustment buttons 42t and 42w (command-input images), and magnification factors available for image enhancement 43. The enlarged-area location map 41, the zooming-adjustment buttons 42t and 42w, and the magnification factors available for image enhancement 43 are displayed in a first area 45, second areas 46t and 46w, and a third area 47, respectively, in the zooming-adjustment command-input picture. When displaying the zooming-adjustment command-input picture, the locations of the first, second, and third areas 45, 46t and 46w, and 47 in the touch-panel monitor 26 are arranged as shown in FIG. 4.

The enlarged-area location map 41 indicates the location of the area of enlargement to be displayed, hereinafter referred to as the target area, within an entire image captured by the effective pixel area of the imaging device 34. The location of the target area is indicated by displaying a frame 44 of the target area within the entire image. Also, an image signal is sent from the second signal-processing circuit 23 to the touch-panel controller 28. The entire image included in the enlarged-area location map 41 is generated based on the received image signal. Accordingly, the user can recognize the location and the size of the target area in the entire image included in the enlarged-area location map 41. In addition, as described later, by touching on any area within the first area, the location of the target area can be changed.

The zooming-adjustment buttons comprise a tele-button 42t and a wide-button 42w.

The tele-button 42t is displayed in a tele-button area 46t. When the tele-button area 46t is touched, a zoom-in operation commences. In the zoom-in operation, the magnification of the enlarged image is increased by a factor of 0.1 per a predetermined time interval. When the tele-button area 46t is touched during the course of the zoom-in operation, the zoom-in operation stops. Also, the zoom-in operation automatically stops when the magnification of the enlarged image reaches an upper limit.

The wide-button 42w is displayed in a wide-button area 46w. When the tele-button area 46t is touched, a zoom-out operation commences. In the zoom-out operation, the magnification factor of the enlarged image is decreased by 0.1 per the predetermined time interval. When the wide-button area 46w is touched during the course of the zoom-out operation, the zoom-out operation stops. Also, the zoom-out operation automatically stops when the magnification factor of the enlarged image reaches a lower limit.

As shown in FIG. 1, a plurality of scope buttons 32 are mounted on a control body 31 of the electronic endoscope 30. Each of the scope buttons 32 is assigned various functions of the endoscope system 10. The function which a scope button 32 is assigned can be designated in a function-assignment menu picture which is linked to the menu-command input picture.

When the user touches on the scope button 32 after it has been assigned a zoom-in/-out function while using the endoscope processor 20, an enlarged image magnified to a designated magnification factor is displayed. When the scope button 32 is touched again, the displayed image is changed to the entire image. In addition, the magnification factor of the enlarged image can be designated by the user's command input to the third area.

When the user touches any area on the touch panel 26 while the zooming-adjustment command-input picture 40 is displayed, the touch-panel controller 28 recognizes which area in the zooming-adjustment command-input picture 40 the user has touched.

If the touched location corresponds to the second areas 46t or 46w, a zooming-adjustment signal is sent to the system controller 22. The system controller 22 calculates the magnification factor of the enlarged image based on the zooming-adjustment signal. A magnification signal corresponding to the calculated magnification factor is sent from the system controller to the enlargement-processing circuit 24. The enlargement-processing circuit 24 generates the enlarged image from the image signal by enlarging a part of the entire image by the magnification factor corresponding to the received magnification signal. In addition, the magnification signal is also sent to the touch-panel controller 28. The touch-panel controller 28 changes the size of the frame 44 of the target area in the enlarged-area location map 41 based on the magnification signal. The initial location of the center of the frame 44 corresponds to the center of the enlarged-area location map 41 that is the center of the first area 45.

If the touched location corresponds to the first area 45, the touch-panel controller 28 determines whether the touched location is in a permission area 48. The permission area 48 is based on the magnification factor of the enlarged image, described later, and used for keeping the target area within the first area 45.

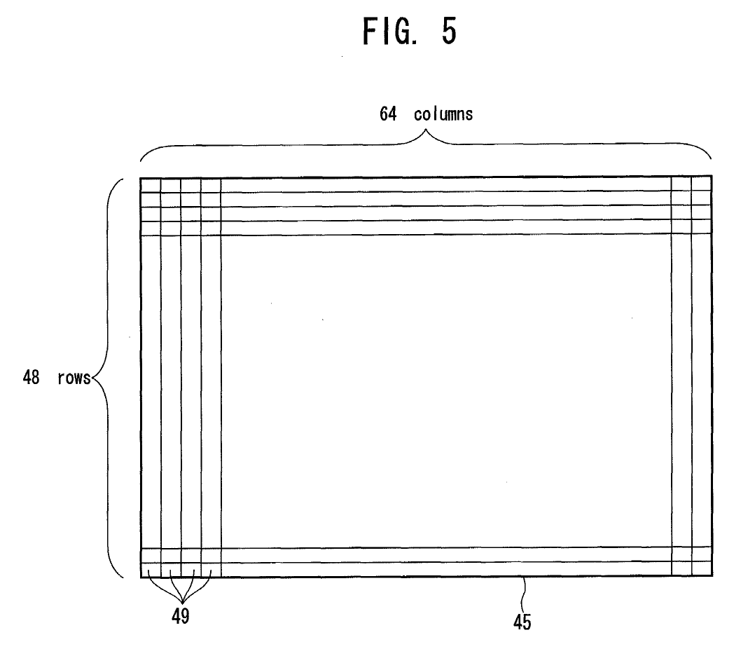

The chosen magnification factor determines the coordinates of corner points P1, P2, P3, and P4 of the bounding box for the frame 44. This rectangular area with corners P1, P2, P3, and P4 is designated the permission area 48, beyond which the center of the enlargement area may not be located. Also, in this embodiment, the touched location is detected from one of unit areas 49. As shown in FIG. 5, the first area is divided into a grid of unit areas 49 consisting of forty eight rows by sixty four columns.

If the touched location is within the permission area 48, the touch-panel controller 28 renews the enlarged-area location map 41 so that the center of the frame 44 corresponds to the touched location. In the renewal, the frame 44 moves within the enlarged-area location map 41. On the other hand, if the touched location is out of the permission area 48, the unit area 49 in the permission area 48 which is nearest to the actually-touched location is selected as the apparently-touched location and treated as the actually touched location. The touch-panel controller 28 renews the enlarged-area location map 41 so that the center of the frame 44 coincides with the apparently-touched location.

When the touched location is within the first area and the frame 44 is to be moved, the touch-panel controller 28 generates a frame-location signal corresponding to the location of the frame 44 to be moved and sends the frame location signal to the enlargement-processing circuit 24 via the system controller 22.

The enlargement-processing circuit 24 generates an enlarged image whose center coincides with the touched location based on the frame-location signal. If the actually-touched location is near an edge of an entire image, a complete enlarged image whose center coincides with the actually-touched location will not fit and is not generated. Thus, the permission area 48 defines the effectively available range for locating the center of the enlarged image.

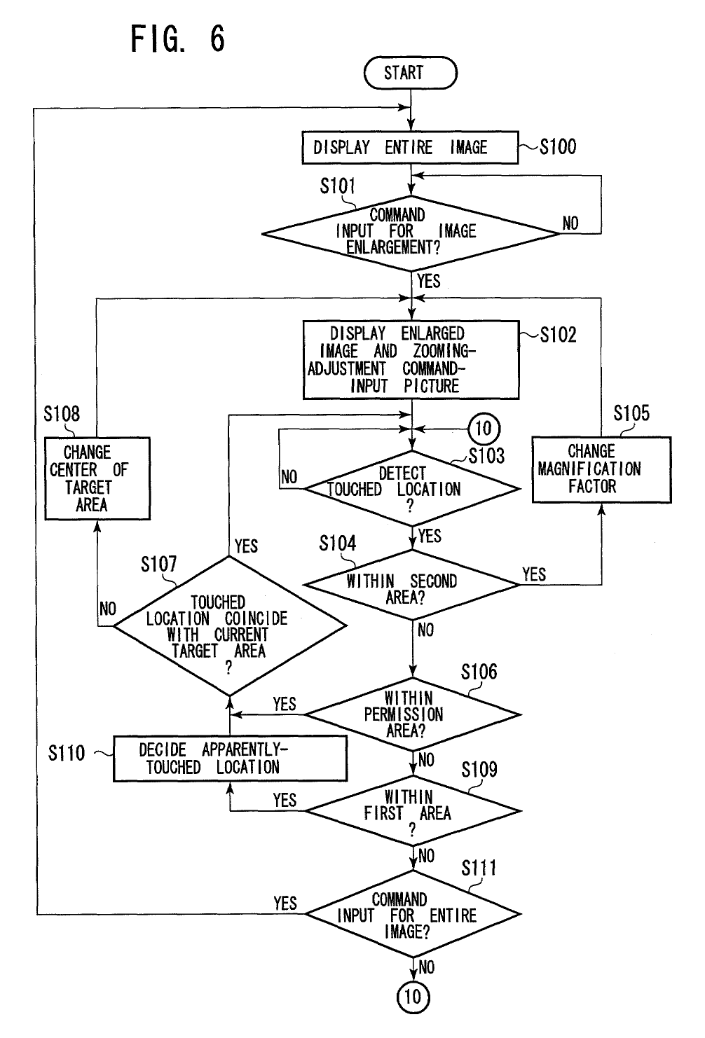

Next, the image-displaying processing that the endoscope processor 20 carries out for displaying a captured image of an object is explained below, using the flowchart of FIG. 6. The image displaying processing starts when an endoscope processor 20 is switched on and an operation mode of the endoscope processor 20 is changed to a mode for displaying an image of a subject. In addition, the image displaying processing finishes when the endoscope processor 22 is switched off or the operation mode of the endoscope processor 20 is changed.

At step S100, the generated image signal is sent to the monitor 11 without undergoing enlargement processing. Then, an entire image is displayed on the monitor 11. At step S101, it is determined whether there is a command input for displaying an enlarged image.

If there is no input command for displaying an enlarged image, step S101 is repeated until a command for displaying an enlarged image is input, and an entire image is kept displayed. If there is an input command for displaying an enlarged image, the process proceeds to step S102.

At step S102, enlargement processing is carried out on an image signal based on a designated magnification factor and the location of the target area, and an enlarged image is displayed on the monitor 11. In addition, the zooming-adjustment command-input picture is ordered to be displayed on the touch-panel monitor 26.

The enlarged-area location map 41 in the zooming-adjustment command-input picture is generated based on the designated magnification factor and the location of the target area. Also, the default magnification factor and location of the target area are set to 1.5 and the center of the entire image, respectively. In addition, the user can change the default magnification factor and location of the target area.

At step S103, it is determined whether the user has touched the touch-panel monitor 26 and the touched location is detected. If a touched location is not detected, step S103 is repeated. If a touched location is detected, the process proceeds to step S104.

At step S104, it is determined whether the detected touched location is within the second areas 46t and 46w. If the touched location is within the second areas 46t and 46w, the process proceeds to step S105. If the touched location is out of the second area 46t, 46w, the process proceeds to step S106.

At step S105, the magnification factor is calculated and the calculated magnification factor is designated as the magnification factor for enlargement. After designation, the process returns to step S102. Then, the enlargement processing is carried out based on the newly designated magnification factor, and the enlarged image is renewed, and the size of the frame 44 in the enlarged-area location map 41 is changed in accordance with the designated magnification factor. Furthermore, the greater the designated magnification factor, the smaller the relative size of the frame 44 in the first area 45 is.

At step S106, it is determined whether the touched location is within the permission area 48 in the enlarged-area location map 41. If the touched location is within the permission area 48, the process proceeds to step S107. On the other hand, if the touched location is out of the permission area 18, the process proceeds to step S109.

At step S107, it is determined whether the touched location coincides with the center of the current target area. If the touched location coincides with the center of the current target area, the process returns to step S103, and the enlarged image is kept displayed without changing the location of the current target area. If the touched location falls outside of the center of the current target area, the process proceeds to step S108.

At step S108, the latest touched location is designated as the center of a new target area. After designation, the process returns to step S102, and an enlarged image with a new center is displayed on the monitor 11, and the enlarged-area location map 41 with frame 44 is displayed on the touch-panel monitor 26.

As described above, if at step S106 the touched location is determined to be out of the permission area 18, the process proceeds to step S109. At step S109, it is determined whether the touched location is within the first area 45. If the touched location is within the first area 45, the process proceeds to step S110. On the other hand, if the touched location is outside the first area 45, the process proceeds to step S111.

At step S110, the unit area 49 in the permission area 48 which is nearest to the actually-touched location is chosen as the apparently-touched location. When the apparently-touched location is chosen, the process proceeds to step S107. Then, some operations are carried out treating the apparently-touched location as a actually-touched location.

At step S111, it is determined whether there is a command input for displaying an entire image. If there is a command input for displaying an entire image, the process returns to step S100. On the other hand, if there is no command input for displaying an entire image, the process proceeds to step S103.

In the above embodiment, the user can change the location of a target area, which is displayed with enlargement in an entire image, by inputting a command to the touch-panel monitor 26 while watching the entire image on the touch-panel monitor 26. Accordingly, the user can easily check the magnification factor and change the location of the target area without watching the monitor 11.

In the above embodiment, the enlarged-area location map 41 includes an image captured by the imaging device 34. However, the captured image may not be included. Even if only the location of the target area in the entire image is displayed on the touch-panel monitor, this endoscope processor is more convenient than those of a prior art. Of course, if the captured image is also superimposed, an endoscope processor such as the above embodiment is even more convenient.

In the above embodiment, the zooming-adjustment command-input picture 40 includes the zooming-adjustment buttons 42t and 42w, and the magnification factors available for image enhancement 43. However, these need not be included. The location of the target area can easily be changed without including the zooming-adjustment buttons 42t and 42w, or the magnification factors available for image enhancement 43.

In the above embodiment, the user's command input is recognized when the touch-panel monitor 26 is touched. However, any other input method for a touch-panel monitor, such as pointing to an area of the touch-panel monitor, could be adapted. There are many kinds of touch-panel monitors, such as capacitive touch-panel monitors, optical-imaging touch-panel monitors, surface acoustic wave touch-panel monitors, and resistive touch-panel monitors. The user's command input may be recognized when a behavior adequate for a selected touch-panel monitor is carried out.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2006-284970 (filed on Oct. 19, 2006), which is expressly incorporated herein, by reference, in its entirety.

1. An endoscope processor that displays an image of a target area with enlargement on a monitor, said target area being a part of an entire image captured by an electronic endoscope, said endoscope processor comprising:
a touch-panel monitor;
a location detector that detects an input location, said input location being a location where the user's input operation is done on said touch-panel monitor;
a touch-panel image generator that orders a target-area location window to be displayed on said touch-panel monitor, said target-area location window indicating the location of said target area in said entire image; and
a location-changer that changes said location of said target area based on said input location, said input location being detected by said location detector when said target-area location window is displayed on said touch-panel monitor.

2. An endoscope processor according to claim 1, wherein when said location-changer changes said location of said target area, said touch-panel image generator renews said target-area location window based on said changed location of said target area.

3. An endoscope processor according to claim 1, wherein
said touch-panel image generator orders a command-input image with said target-area location window to be displayed on said touch-panel monitor, said command-input image used for changing the magnification factor used to enlarge an image of said target area, and
said magnification factor is changed when said input location detected by said location detector agrees with said command-input image displayed on said touch-panel monitor.

4. An endoscope processor according to claim 1, wherein said entire image is displayed on said touch-panel monitor and said target-area location window is said displayed entire image where said location of said target area is indicated.

5. An endoscope processor according to claim 1, wherein said location-changer changes said location of said target area so that the center of said target area agrees with said input location.

6. An endoscope processor according to claim 1, wherein it is permitted to change said location of said target area within a permission area, the extent of said permission area being decided according to the magnification factor used to enlarge an image of said target area.

7. An endoscope system, comprising:
an electronic endoscope that captures a subject;
an endoscope processor that enlarges an image of a target area being a part of an entire image captured by said electronic endoscope, said endoscope processor having a touch-panel monitor, a location detector, a touch-panel image generator, and a location-changer, said location detector detecting an input location being a location where the user's input operation is done on said touch-panel monitor, said touch-panel image generator ordering a target-area location window to be displayed on said touch-panel monitor, said target-area location window indicating the location of said target area in said entire image, said location-changer changing said location of said target area based on said input location detected by said location detector when said target-area location window is displayed on said touch-panel monitor; and
a monitor where said enlarged image of said target area is displayed.
